(19) United States
(12) Patent Application Publication
(10) Pub. No.: US 2024/0277510 A1
(43) Pub. Date: Aug. 22, 2024

(54) CUSTOMIZED INTEGRATED OSTOMY POUCH COMFORT PANEL AND POUCH COVER AND METHOD OF MAKING SAME

(71) Applicant: Hollister Incorporated, Libertyville, IL (US)

(72) Inventors: Mark W. Jockel, Vernon Hills, IL (US); Jesse E. Menoni, Lake Zurich, IL (US)

(21) Appl. No.: 18/681,186

(22) PCT Filed: Sep. 13, 2022
(86) PCT No.: PCT/US2022/043316

§ 371 Date: Feb. 5, 2024

Related U.S. Application Data
(60) Provisional application No. 63/244,509, filed on Sep. 15, 2021.

(51) Int. Cl. A61F 5/445; A61F 5/44; G06T 7/90

(52) U.S. Cl. CPC A61F 5/445; A61F 5/4404; G06T 7/90; G06T 2207/10024; G06T 2207/30004

(57) ABSTRACT

A method of manufacturing at least one of an ostomy pouch cover and an integrated ostomy comfort panel is disclosed herein. The method can comprise receiving a color image depicting a subject and a calibrated color gauge. The color image can be processed with the calibrated color gauge to identify a color for the subject. The color can correspond to a matched color selected from a population of predetermined colors. The color of the subject can be printed and applied to at least a portion of a surface of at least one of the ostomy pouch cover and the integrated ostomy comfort panel. An ostomy pouch cover and an ostomy pouch having an integrated comfort panel with printing applied thereto is further provided according to methods presented herein.

### BACKGROUND

The present disclosure relates to ostomy appliances, and more particularly to customized integrated comfort panels for an ostomy pouch and customized pouch covers. The present disclosure further relates to methods for making customized ostomy pouch comfort panels and ostomy pouch covers, and more particularly printing custom colors or designs thereon.

Ostomy pouches for the collection of body waste output from a stoma are well known. Generally, ostomy pouches have side panels with an exterior skin-contacting surface. For user comfort, certain pouches incorporate an integrated comfort panel along the exterior layer of the side panels which can be comprised of fabric or other soft material to reduce irritation to a user's skin and make the pouch more comfortable to wear.

Out of a desire for discreteness, users of ostomy pouches have commonly expressed a preference for ostomy pouches that are opaque or non-translucent to conceal waste contents within the pouch. Users also commonly employ separate ostomy pouch covers which can be placed over the ostomy pouch to conceal the underlying pouch. Frequently, to further make the ostomy pouch seem more subtle or inconspicuous, certain users can prefer that the pouch or pouch cover be colored to closely match the user's skin tone and complexion so as to camouflage the pouch against the user's skin. Other users can prefer that the pouch or pouch cover have a unique or distinctive decorative appearance to suit their tastes and desires.

Skin tone color guides, such as for example the SkinTone™ Guide offered by Pantone LLC (at https://www.pantone.com/articles/product-spotlight/skintone-guide-revealing-the-new-pantone-skintone-guide), are generally known and can be particularly useful in applications where it is important to create or adapt an article to have a color that matches the color of a particular human skin tone. Due to the fact that there are numerous different types of human skin tones, Pantone's SkinTone™ Guide, is regarded as being particularly advanced in that it was formulated to match and replicate the closest physical representations of skin colors, and thus comprises a collection of one hundred ten real skin tone shades.

As will be appreciated, although such skin color matching capabilities are generally useful, with specific regard to the manufacture of prefabricated off the shelf ostomy pouches, it would be commercially and logistically impracticable for a manufacturer of such products to make and offer commercially reasonable quantities of every type and style of ostomy pouch in one hundred ten different colors.

For at least the above-noted reasons there has remained a need in the art to develop a method of custom manufacturing ostomy pouches and ostomy pouch covers to closely match the user's skin type or to include customized decorative elements to correspond to the individual's desired tastes and preferences. There has further remained a need in the art for a method of custom coloring the integrated comfort layer of ostomy pouches to suit a user's specific needs and tastes. A need has also remained for customizable integrated ostomy pouch comfort panels and ostomy pouch covers manufactured according to methods embodied herein.

### SUMMARY OF THE DISCLOSURE

Embodiments presented herein are directed to a method of manufacturing at least one of an ostomy pouch cover and an integrated ostomy comfort panel. According to exemplary embodiments such methods can comprise receiving a color image depicting a subject and a calibrated color gauge. The color image can be processed with the calibrated color gauge to identify a color for the subject. The color can correspond to a matched color selected from a population of colors. The color of the subject can be printed and applied to at least a portion of a surface of at least one of the ostomy pouch cover and the integrated ostomy comfort panel.

According to exemplary embodiments, at least a portion of a person's skin can be the subject according to the methods presented herein. The color of the subject can be printed and applied directly onto at least a portion of the surface of at least one of the ostomy pouch cover and the integrated ostomy comfort panel. A direct to textile printer can be utilized for printing the color of the subject directly onto at least a portion of the surface of at least one of the ostomy pouch cover and the integrated ostomy comfort panel. Alternatively, the color of the subject can be printed and applied onto a transfer media and the color of the subject can be laminated from the transfer media onto at least a portion of the surface of at least one of the ostomy pouch cover and the integrated ostomy comfort panel. A heated laminator can be utilized for laminating the color of the subject from the transfer media onto at least a portion of the surface of at least one of the ostomy pouch cover and the integrated ostomy comfort panel.

According to exemplary embodiments, instructions for capturing the color image of the subject together with the calibrated color gauge and transmitting the image to a third party for processing can be provided. Additionally, the color image with the calibrated color gauge can be processed by detecting color characteristics of both the subject and colors presented on the calibrated color gauge and by matching said color characteristics with corresponding color characteristics from colors in the population of predetermined colors. At least one of the colored image and color can be stored to facilitate printing and applying the color of the subject to the surfaces of articles in the future.

Embodiments presented herein are also directed to a method of manufacturing at least one of an ostomy pouch cover and an integrated ostomy comfort panel whereby a color image depicting a calibrated color gauge and a subject having at least one of an ornamental feature and a graphic character is received. The color image with the calibrated color gauge can be processed to identify a color for at least one of the ornamental feature and the graphic character. The color corresponding to a matched color can be selected from a population of predetermined colors. At least a portion of at least one of the ornamental feature and the graphic character including the color therefor can be printed and applied to at least a portion of a surface of at least one of the ostomy pouch cover and the integrated ostomy comfort panel.

According to exemplary embodiments, at least one of the ornamental feature and the graphic character including the color therefor can be printed and applied directly onto at least a portion of the surface of at least one of the ostomy pouch cover and the integrated ostomy comfort panel. A direct to textile printer can be utilized for printing at least one of the ornamental feature and the graphic character including the color therefor directly onto at least a portion of the surface of at least one of the ostomy pouch cover and the integrated ostomy comfort panel. Alternatively, at least one of the ornamental feature and the graphic character including the color therefor can be printed and applied onto a transfer media and at least one of the ornamental feature and the graphic character including the color therefor can be laminated from the transfer media onto at least a portion of the surface of at least one of the ostomy pouch cover and the integrated ostomy comfort panel. A heated laminator can be utilized for laminating at least one of the ornamental feature and the graphic character including the color therefor from the transfer media onto at least a portion of the surface of at least one of the ostomy pouch cover and the integrated ostomy comfort panel. According to exemplary embodiments, instructions for capturing the color image of the calibrated color gauge and the subject can be provided and for transmitting the image to a third party for processing.

Embodiments presented herein are also directed to an ostomy pouch. According to exemplary embodiments, the ostomy pouch can comprise a body-side wall and a distal-wall joined at an outer periphery and defining an interior volume comprising a collection area, an inlet for receiving ostomy effluent, and a comfort panel attached to an outer surface of at least one of the body-side wall and distal wall, wherein the comfort panel can have printing thereon. The printing can comprise at least one of a color, an ornamental feature and a graphic character. The printing can be applied to at least a portion of the comfort panel by receiving a color image depicting a calibrated color gauge and a subject comprising at least one of an original color, an original ornamental feature and an original graphic character printed thereon. The color image can be processed with the calibrated color gauge to identify color characteristics of at least one of the original color, the original ornamental feature and the original graphic character and at least one printable color can be selected which corresponds to the identified color characteristics. The printable color can be selected from a population of predetermined printable colors. At least one of the color, the ornamental feature and the graphic character including the at least one printable color can be printed and applied along at least the portion of the comfort panel.

According to exemplary embodiments the subject can be at least a portion of a person's skin according to methods presented herein. Additionally, the portion of the comfort panel with the printing applied thereto can be at least one of an outside surface, an inside surface, a front surface and a back surface of the comfort panel.

Embodiments presented herein can also be directed to an ostomy pouch cover. According to exemplary embodiments, the ostomy pouch cover can comprise a cover material having printing thereon. The printing can comprise at least one of a color, an ornamental feature and a graphic character. The printing can be applied to at least a portion of the cover material by receiving a color image depicting a calibrated color gauge and a subject comprising at least one of an original color, an original ornamental feature and an original graphic character printed thereon. The color image can be processed with the calibrated color gauge to identify color characteristics of at least one of the original color, the original ornamental feature and the original graphic character. At least one printable color corresponding to the identified color characteristics can be selected from a population of predetermined printable colors. At least one of the color, the ornamental feature and the graphic character including the at least one printable color can be printed and applied on the portion of the cover material.

Other objects, advantages and features of the present disclosure will be understood and appreciated by persons of ordinary skill in the art from consideration of the following specification taken in conjunction with the accompanying drawings.

### BRIEF DESCRIPTION OF THE DRAWINGS

FIG. 1 is a graphical flowchart of a method according to exemplary embodiments presented herein.

FIG. 2 is a graphical flowchart of a method according to exemplary embodiments presented herein.

FIG. 3 is a schematic perspective view of an ostomy pouch with a customized integrated comfort panel according to exemplary embodiments presented herein.

FIG. 4 is a schematic perspective view of an ostomy pouch cover according to exemplary embodiments presented herein.

### DETAILED DESCRIPTION

While the present disclosure is susceptible of embodiment in various forms, there is shown in the drawings and will hereinafter be described a presently preferred embodiment with the understanding that the present disclosure is to be considered an exemplification and is not intended to limit the disclosure to the specific embodiment illustrated.

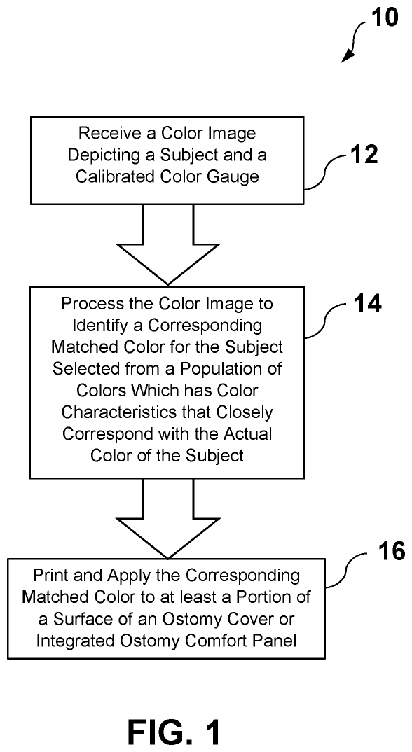

With reference now to the accompanying figures, and FIG. 1 in particular, an exemplary method of manufacturing 10 an ostomy pouch cover and/or an integrated ostomy comfort panel is representatively shown. As shown schematically in FIG. 1, method 10 can comprise receiving 12 a color image that depicts a subject and a calibrated color gauge. According to exemplary embodiments, the color image can be received from an individual in connection with an order or purchase for a custom printed ostomy pouch cover or ostomy pouch having a customized skin-protecting comfort panel. The image can be a digitized image or photograph captured by means for digital photography and either stored in digital format as a computer file or in physical form printed on paper. The image or photograph can also be captured via analog photography on paper, film or other media.

According to exemplary embodiments, the calibrated color gauge or color matching tool can be, for example, of a type similar to color match cards such as those provided by Pantone LLC (as shown at https://www.pantone.com/products/color-capture-devices/pantone-color-match-card) or Edmund Optics Inc. (available at https://www.edmundoptics.com/p/colorgauge-micro-matte/29792/), the details and specifications of which are hereby incorporated by reference as if set forth fully herein. It will be understood however, that such color match cards are referenced for exemplification only and that other calibrated color gauges and color matching tools and processes can be provided or utilized in connection with embodiments presented herein without limiting the scope of the subject invention.

As is conventionally known with color matching processes, a calibrated color gauge or tool can generally comprise a multicolored array or test pattern of standard reference colors having particular color characteristics (e.g. hue, saturation, and intensity or brightness). During the color matching process, an image depicting the calibrated color gauge together with a subject can be analyzed and processed by software which is able to detect variances in the color characteristics of the standard reference colors of the test pattern. Such variances can be due to a number of factors including for example the camera settings, the angle at which the image was taken, the finish of certain materials and/or ambient conditions that exist in the location where the image was taken, such as for example, the use of a camera flash or the overall lighting conditions. Upon analysis of the variations of the reference colors from the test pattern, the system can model the same variances to the colors of the subject in the image. In carrying out this analysis, the color matching system is able to “color correct” the colors of the subject to identify normalized or matching colors from a population of predetermined colors which best match the actual colors of the subject.

According to exemplary embodiments presented herein, the subject of the image can be a person, or at least a portion of a person's body which depicts the person's skin tone or color. The subject can also be other articles, objects or works as desired including inanimate articles having colors, ornamental features such as patterns, shapes, textures or images, or graphic characters such as letters, words, names, numbers or messages without limitation.

As shown in FIG. 1, method 10 according to exemplary embodiments can comprise utilizing a color matching system and software for processing 14 the color image with the subject and calibrated color gauge to identify a normalized or matched color for the subject which can be selected from a population of colors. Accordingly, it will be understood that the matched color can account for variations to color characteristics shown in the image and can embody a color that represents the closest match to the color of the actual subject.

Method 10 illustrated in FIG. 1 can further comprise printing and applying 16 the corresponding matched color to at least a portion of a surface of at least one of the ostomy pouch cover and the integrated ostomy comfort panel. According to exemplary embodiments, depending on the particular fabric of the ostomy pouch cover or comfort panel, the corresponding matched color can be directly printed, applied thereon by a direct to textile printer (DDT) such as for example, the Mutoh ValueJet 1938TX printer or similar or alternatively via water transfer printing hydrographics wherein a water-soluble polyvinyl alcohol hydrographic film with the matched color gravure-printed thereon is applied to the ostomy pouch cover with water and an activator solution whereby the ink in the matched color adheres to the surface of the ostomy pouch cover. It will be understood however that other printing machines or equipment can be used without limitation without departing from the scope of the subject invention. Alternatively, if needed or desired, the corresponding matched color can be first printed or applied to a transfer media. A heated laminator can then be used to laminate the color from the transfer media onto the integrated ostomy comfort panel or ostomy pouch cover. The printing and transfer process can be in-line or off-line to ostomy pouch assembly machines. Further, the custom color(s) can be printed on the outside, inside, front and/or back of the ostomy comfort panel (all four sides) or on the inside or outsides of the ostomy pouch cover.

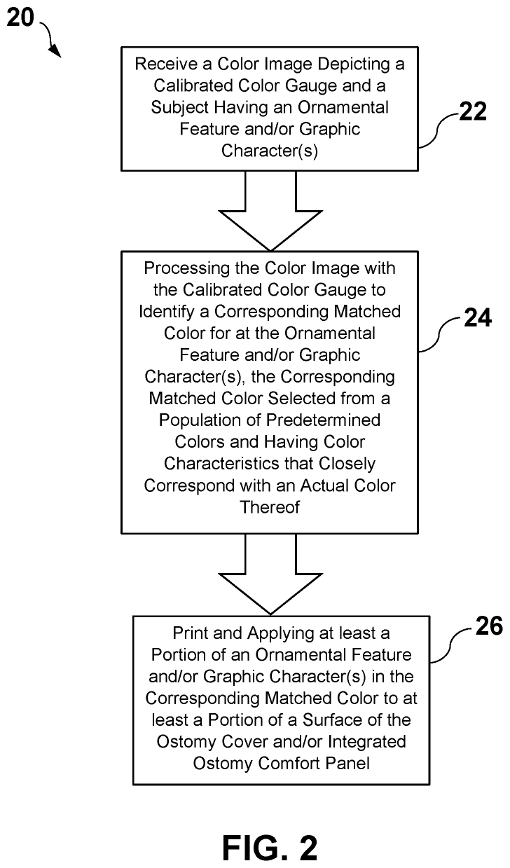

FIG. 2 illustrates another method 20 for manufacturing an ostomy pouch cover or ostomy pouch comfort panel according to embodiments presented herein. As shown schematically in FIG. 2, method 20 can comprise receiving 22 a color image depicting a calibrated color gauge and a subject having an ornamental feature and/or graphic character(s) displayed thereon. It will be understood that the color image, color gauge and the subject can be or have characteristics of the type described above with reference to the embodiment of FIG. 1. It will also be recognized and understood that the ornamental feature can comprise designs, patterns, shapes or other artistic works and that the graphic characters can include letters, words, names, numbers or messages without limitation. According to exemplary embodiments shown schematically in FIG. 2, the color image with the subject and calibrated color gauge can be processed 24 using a color matching system and software to identify a normalized or matched color for the ornamental feature and/or graphic characters. As described above, the corresponding matched color can account for variations to color characteristics shown in the image and can embody a color that represents the closest match to the color of the actual ornamental design or graphic characters of the subject.

According to exemplary embodiments shown schematically in FIG. 2, the ornamental feature and/or graphic characters in the corresponding matched color can be printed or applied 26 to at least a portion of a surface of at least one of the ostomy pouch cover or the integrated ostomy comfort panel. According to exemplary embodiments, depending on the particular fabric of the ostomy pouch cover or comfort panel, the ornamental feature and/or graphic characters in the corresponding matched color can be directly printed or applied onto the ostomy pouch cover or integrated comfort panel by a direct to textile printer (DDT) such as for example, the Mutoh ValueJet 1938TX printer or similar or alternatively via water transfer printing hydrographics wherein a water-soluble polyvinyl alcohol hydrographic film with the matched color gravure-printed thereon is applied to the ostomy pouch cover with water and an activator solution whereby the ink in the matched color adheres to the surface of the ostomy pouch cover. It will be understood however that other printing machines or equipment can be used without limitation without departing from the scope of the subject invention. Alternatively, if needed or desired, the ornamental feature and/or graphic characters in the corresponding matched color can be first printed or applied to a transfer media. A heated laminator can then be used to laminate the ornamental feature and/or graphic characters in the corresponding matched color from the transfer media onto the integrated ostomy comfort panel or ostomy pouch cover. The printing and transfer process can be in-line or off-line to ostomy pouch assembly machines. Further, the ornamental feature and/or graphic characters in the corresponding matched color(s) can be printed on the outside, inside, front and/or back of the ostomy comfort panel (all four sides) or on the inside or outsides of the ostomy pouch cover.

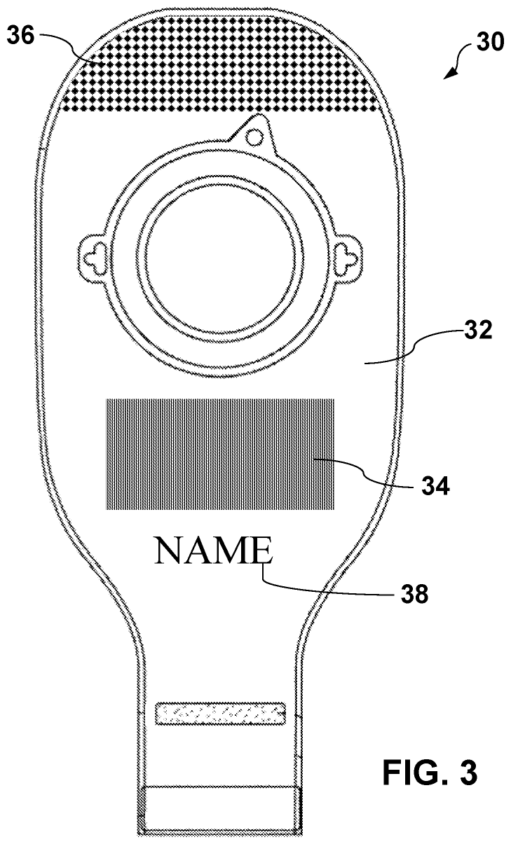

FIG. 3 schematically illustrates an ostomy pouch 30 having a customized integrated comfort panel 32. As shown in FIG. 3, integrated comfort panel 32 can be integrated along a side panel of ostomy pouch 30 and can be comprised of a flexible laminated material having a skin-contacting region comprised of soft fabric or other material to reduce irritation to a user's skin and make the pouch more comfortable to wear. For example, comfort panel 32 may be attached to a body-side surface and/or distal-side surface of the ostomy pouch 30. The comfort panel 32 may be attached to a pouch wall via an adhesive or heat sealed along the periphery of the ostomy pouch 30. According to exemplary embodiments shown in FIG. 3, comfort panel 32 can be customized according to embodiments disclosed herein and can have printing thereon. As shown in FIG. 3, the printing can comprise a color 34, ornamental feature 36 and/or graphic character(s) 38. From the foregoing, it will be understood that such printing can be printed and/or applied from an image processed with color match technology which accounts for variations to color characteristics shown in the image to identify a corresponding matched color that represents the closest match to the color of the actual color, ornamental design or graphic characters of the subject.

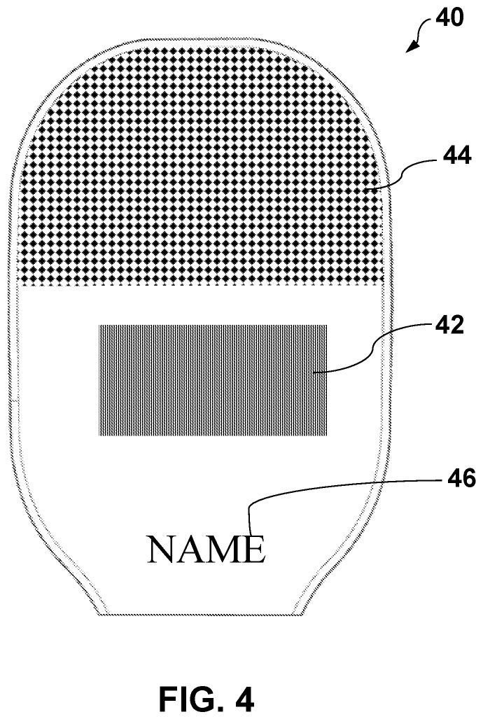

FIG. 4 schematically illustrates a customized ostomy pouch cover 40 according to exemplary embodiments presented herein. Ostomy pouch cover 40 may be configured to cover an ostomy pouch and may be provided in various sizes and shapes. As shown in FIG. 3, ostomy pouch cover 40 can be comprised of a flexible material such as soft fabric or other material to reduce irritation to a user's skin and make the pouch more comfortable to wear. According to exemplary embodiments shown in FIG. 4, ostomy pouch cover 40 can be customized according to embodiments disclosed herein and can have printing thereon. As shown in FIG. 4, the printing can comprise a color 42, ornamental feature 44 and/or graphic character(s) 46. From the foregoing, it will be understood that such printing can be printed and/or applied from an image processed with color match technology which accounts for variations to color characteristics shown in the image to identify a corresponding matched color that represents the closest match to the color of the actual color, ornamental design or graphic characters of the subject.

For the present disclosure, it will be appreciated that embodiments presented herein can be particularly desirable by users of ostomy appliances by enabling improved customization of ostomy appliances by facilitating more accurate color matching, including close matching capability to a desired skin tone. It will be further appreciated that embodiments presented herein can be particularly useful to manufactures by facilitating more color customization to prefabricated off-the-shelf ostomy appliances to accommodate a wider variety of tastes and needs of customers.

In the present disclosure, the words “a” or “an” are to be taken to include both the singular and the plural. Conversely, any reference to plural items shall, where appropriate, include the singular. All of the concentrations noted herein as percentage are percent by weight unless otherwise noted.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

1. A method of manufacturing at least one of an ostomy pouch cover and an integrated ostomy comfort panel comprising:
receiving a color image depicting a subject and a calibrated color gauge;
processing the color image with the calibrated color gauge to identify a corresponding matched color for the subject, the corresponding matched color being selected from a population of predetermined colors and having color characteristics that substantially correspond with an actual color of the subject, and
printing and applying the corresponding matched color to at least a portion of a surface of at least one of the ostomy pouch cover and the integrated ostomy comfort panel.

2. The method of claim 1 wherein the subject is at least a portion of a person's skin.

3. The method of claim 1 wherein the printing and applying comprises printing the corresponding matched color directly onto at least a portion of the surface of at least one of the ostomy pouch cover and the integrated ostomy comfort panel.

4. The method of claim 3 wherein a direct to textile printer is utilized for printing the corresponding matched color directly onto at least a portion of the surface of at least one of the ostomy pouch cover and the integrated ostomy comfort panel.

5. The method of claim 1 wherein the printing and applying comprises printing the corresponding matched color onto a transfer media and laminating said color from the transfer media onto at least a portion of the surface of at least one of the ostomy pouch cover and the integrated ostomy comfort panel.

6. The method of claim 5 wherein a heated laminator is utilized for laminating the corresponding matched color from the transfer media onto at least a portion of the surface of at least one of the ostomy pouch cover and the integrated ostomy comfort panel.

7. The method of claim 1 further comprising providing instructions for capturing the color image of the subject together with the calibrated color gauge and transmitting the image to a third party for processing.

8. The method of claim 1 wherein the processing of the color image with the calibrated color gauge comprises detecting variances in color characteristics of colors presented on the calibrated color gauge and modeling such variances relative the subject.

9. The method of claim 1 further comprising storing at least one of the color image and corresponding matched color to facilitate printing and applying the corresponding matched color to the surfaces of articles in the future.

10. The method of claim 1 wherein the printing and applying is carried out via water transfer printing wherein a water-soluble hydrographic film with the matched color printed thereon is applied to the ostomy pouch cover with water and an activator solution.

11. A method of manufacturing at least one of an ostomy pouch cover and an integrated ostomy comfort panel comprising:
receiving a color image depicting a calibrated color gauge and a subject having at least one of an ornamental feature and a graphic character displayed thereon;
processing the color image with the calibrated color gauge to identify a corresponding matched color for at least one of the ornamental feature and the graphic character, said corresponding matched color being selected from a population of predetermined colors and having color characteristics that substantially correspond with an actual color thereof, and
printing and applying at least a portion of at least one of the ornamental feature and the graphic character in the corresponding matched color therefor to at least a portion of a surface of at least one of the ostomy pouch cover and the integrated ostomy comfort panel.

12. The method of claim 10 wherein the printing and applying comprises printing at least one of the ornamental feature and the graphic character including the corresponding matched color therefor directly onto at least a portion of the surface of at least one of the ostomy pouch cover and the integrated ostomy comfort panel.

13. The method of claim 11 wherein a direct to textile printer is utilized for printing at least one of the ornamental feature and the graphic character including the corresponding matched color therefor directly onto at least a portion of the surface of at least one of the ostomy pouch cover and the integrated ostomy comfort panel.

14. The method of claim 10 wherein the printing and applying comprises printing at least one of the ornamental feature and the graphic character including the corresponding matched color therefor onto a transfer media and laminating at least one of the ornamental feature and the graphic character including the said matched color therefor from the transfer media onto at least a portion of the surface of at least one of the ostomy pouch cover and the integrated ostomy comfort panel.

15. The method of claim 13 wherein a heated laminator is utilized for laminating at least one of the ornamental feature and the graphic character including the corresponding matched color therefor from the transfer media onto at least a portion of the surface of at least one of the ostomy pouch cover and the integrated ostomy comfort panel.

16. The method of claim 10 further comprising providing instructions for capturing the color image of the calibrated color gauge and the subject and for transmitting the image to a third party for processing.

17. An ostomy pouch comprising:
a body-side wall and a distal-wall joined at an outer periphery and defining an interior volume comprising a collection area;
an inlet for receiving ostomy effluent;
a comfort panel attached to an outer surface of at least one of the body-side wall and distal wall, the comfort panel having printing thereon, said printing comprising at least one of a color, an ornamental feature and a graphic character;
wherein said printing is applied to at least a portion of the comfort panel by:
receiving a color image depicting a calibrated color gauge and a subject comprising at least one of the color, the ornamental feature and the graphic character printed thereon;
processing the color image with the calibrated color gauge to detect variances in color characteristics of the calibrated color gauge, said variances being modeled against at least one of the color, the ornamental feature and the graphic character, and at least one corresponding matched color being identified, the corresponding matched color being selected from a population of colors and having color characteristics that substantially correspond with an actual color of at least one of the subject, the ornamental feature and the graphic character, and
printing and applying at least one of the corresponding matched color, the ornamental feature and the graphic character including the matched color therefor along at least the portion of the comfort panel.

18. The ostomy pouch of claim 16 wherein the subject is at least a portion of a person's skin.

19. The ostomy pouch of claim 16 wherein the portion of the comfort panel with the printing applied thereto is at least one of an outside surface, an inside surface, a front surface and a back surface of the comfort panel.

20. (canceled)
